(19) United States
(12) Patent Application Publication
(10) Pub. No.: US 2023/0211116 A1
(43) Pub. Date: Jul. 6, 2023

(54) PORTABLE OXYGEN FLOW REGULATOR

(71) Applicant: Deann Fergus, Kelowna (CA)

(72) Inventor: John Terence Fergus, Kelowna (CA)

(21) Appl. No.: 17/901,564

(22) Filed: Sep. 1, 2022

Related U.S. Application Data
(60) Provisional application No. 63/296,839, filed on Jan. 5, 2022.

(51) Int. Cl. A61M 16/20; A61M 16/08; A61M 16/10; A61M 39/10

(52) U.S. Cl. CPC A61M 16/208; A61M 16/0816; A61M 16/1005; A61M 39/1055; A61M 2039/1038

(57) ABSTRACT

An apparatus for controlling the flow of oxygen to a patient comprises an outer housing extending between first and second ends having a passage therethrough. The housing composes a first portion extending from the first end towards the second end and a second portion extending from the second end towards the first end wherein the second portion is threadably rotatable relative to the first portion so as to longitudinally displaced relative to the first portion under rotation thereof so as to adjust at least a portion of the passage extending therethrough adjusting a flow capacity of the passage. The apparatus further comprises connectors at each of the first and second ends operable to connect to oxygen conduits to fluidically conned the oxygen conduits to the passage through the housing.

### BACKGROUND

### 1. Technical Field

This disclosure relates generally to distribution of gasses and in particular to a regulator for the flow of personal oxygen to a patient.

### 2. Description of Related Art

Medical oxygen is commonly utilized for patients having difficulty maintaining a healthy blood oxygen level. The reasons for such difficulty are numerous, but some of such patients are not otherwise required to be kept in the hospital or other facility for monitoring or other care. In such cases, the patient may be provided with a portable oxygen concentrator which allows them to continue with more regular daily living activities.

One difficulty with many portable oxygen concentrators is that the flow rate of the concentrator is set on the device itself. As illustrated in FIG. 1, such devices may not always be readily available such as being located within a backpack or other carrying bag. Accordingly,

### SUMMARY OF THE DISCLOSURE

According to a first embodiment, there is disclosed an apparatus for controlling the flow of oxygen to a patient comprising an outer housing extending between first and second ends having a passage therethrough. The housing comprises a first portion extending from the first end towards the second end and a second portion extending from the second end towards the first end wherein the second portion is threadably rotatable relative to the first portion so as to longitudinally displaced relative to the first portion under rotation thereof so as to adjust at least a portion of the passage extending therethrough adjusting a flow capacity of the passage. The apparatus further comprises connectors at each of the first and second ends operable to connect to oxygen conduits to fluidically connect the oxygen conduits to the passage through the housing.

The second portion may include a swivel and a central portion, wherein the central portion is threadably rotatably connected to the first portion and the swivel includes the connector at the second end of the apparatus. The swivel and the central portion may include the passage extending therethrough. The passage may be sealed at the connection between the swivel and the central portion.

The first portion may include a protrusion sized to be threadably received within the second portion. The protrusion on the first portion may include threading on the exterior thereof. The protrusion may include a conical distal end.

The second portion may include a cavity therein corresponding to and sized to receive the protrusion of the first portion therein. The cavity may include a bottom end having a shape corresponding to the distal end of the protrusion. The passage may extend through the second portion to the cavity. The passage may extend along a central axis of the first portion.

The passage may be obstructed at a distal end of the protrusion proximate to the bottom end of the cavity. The passage may include at least one radially extending passage to an exterior of the protrusion within to the bottom end of the cavity. The at least one radially extending passage is positioned to not overlap with the passage through the second portion. Longitudinal displacement of the first portion relative to the second portion varies the distance between the protrusion at the at least one radially extending passage and the bottom end of the cavity. The first and second connectors may comprise a protrusion sized to be received within an oxygen tube and a threaded collar adapted to secure the oxygen tube thereto.

One of the first or second portions may include at least one ridge extending therefrom adapted to be engaged by a corresponding indexing member extending from the other of the first or second portions.

According to a further embodiment, there is disclosed a method of adjusting airflow to a patient comprising connecting a housing in line with oxygen tubes to the patient, providing the oxygen tubes with a source of oxygen and rotating a second portion of the housing relative to a first portion of the housing to adjust a restriction in a passage extending through the housing.

Other aspects and features of the present disclosure will become apparent to those ordinarily skilled in the art upon review of the following description of specific embodiments in conjunction with the accompanying figures.

### BRIEF DESCRIPTION OF THE DRAWINGS

The accompanying drawings constitute part of the disclosure. Each drawing illustrates exemplary aspects wherein similar characters of reference denote corresponding parts in each view,

FIG. 1 is a perspective view of a patient having an oxygen supply line including a regulator according to a first embodiment of the present disclosure therein.

FIG. 2 is a perspective view of an apparatus for regulating oxygen flow for the patient of FIG. 1 according to a first embodiment of the present disclosure.

FIG. 3 is a cross sectional view of the apparatus of FIG. 2.

FIG. 4 is a perspective view of the apparatus of FIG. 2 according to a further embodiment.

### DETAILED DESCRIPTION

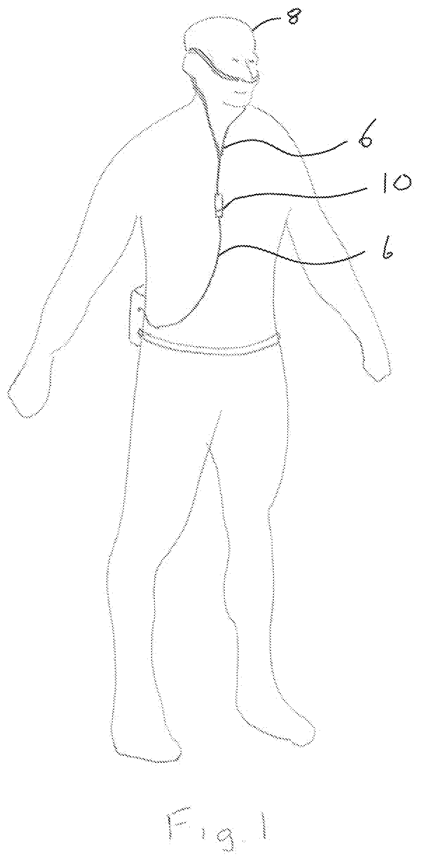

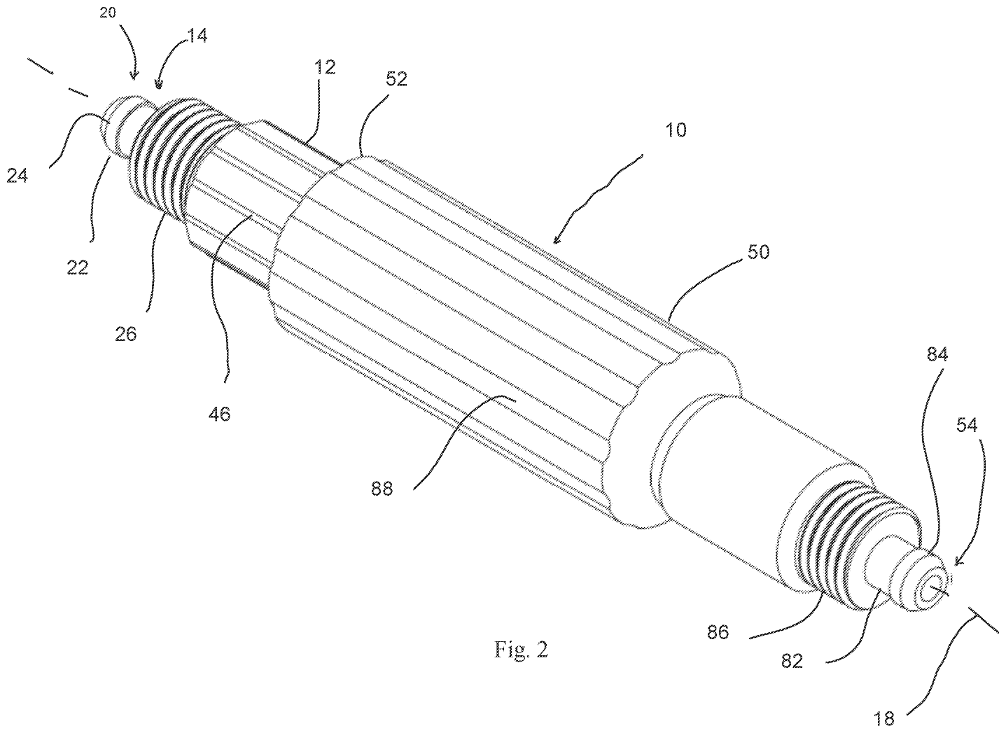

Aspects of the present disclosure are now described with reference to exemplary apparatuses, methods and systems. Referring to FIG. 1, an exemplary apparatus for regulating an oxygen flow rate for a patient 8 according to a first embodiment is shown generally at 10. In particular, the apparatus 10 is located inline with an oxygen tube 6 extending from an oxygen concentrator 4 to the patient as are commonly known. As illustrated in FIG. 2, the apparatus 10 comprises a first or inner body or portion 12 connected to the oxygen tube 6 extending to the oxygen concentrator and a second or outer body 50 connected to the oxygen tube 6 extending to the patient. The second body 50 is rotatable relative to the first body 12 so as to adjust a portion of a passage extending through the apparatus thereby adjusting the permissible flow rate of oxygen therethrough.

The first body 12 comprises an elongate body extending between first and second ends, 14 and 16, respectively along a longitudinal axis 18. The first end 14 includes a connector 20 for connection to a standard oxygen tubing 6 extending from the oxygen concentrator 4. As illustrated, the connector 20 may include a male connector 22 adapted to be received within the tube 6 with an annular ridge 24 therearound and a threaded cylinder 26 adapted to receive a collar 28 (not shown in FIG. 2) on the tube 6. It will also be appreciated that other tube connector types may also be utilized.

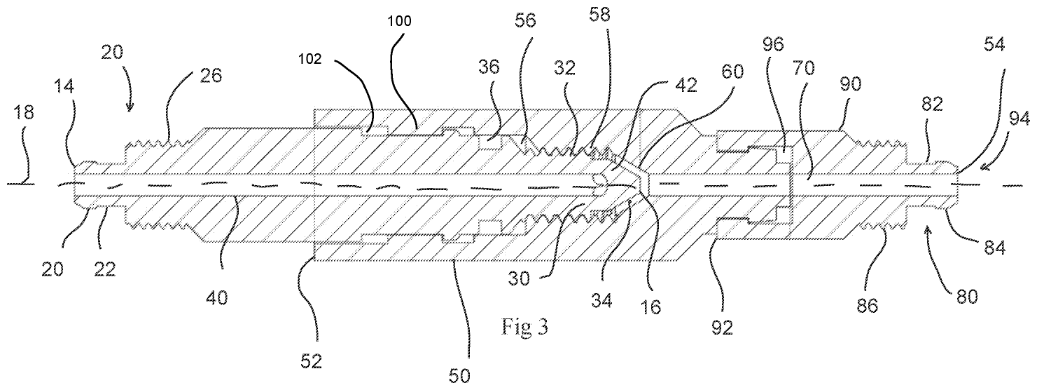

Turning now to FIG. 3, the second end 16 comprises a cylindrical portion 30 sized to be received within the second body 14. The cylindrical portion 30 includes external threading 32 therearound adapted to be engaged with corresponding threading 58 on the interior of the second body 14 as will be more fully described below. The distal end of the first body 12 includes a conical end surface 36 defining a passage between the first and second bodies 12 and 14 which is operable to be narrowed or widened to adjust the flow of oxygen through the apparatus. The first body 12 includes a passage 40 extending along the axis 18 of the apparatus from the first end 14. Proximate to the second end 16, the passage 40 diverges from the axis to one or more radial passage 42 extending to the conical end surfaces. The outer surface of the first body 12 may include ridges, grooves 46 or any other surface treatment to assist gripping by a user.

The second body 50 extends between first and second ends 52 and 54, respectively along the longitudinal axis 18. As illustrated in FIG. 3, the first end includes a cavity 56 thereinto sized to receive the cylindrical portion 30 of the first body 12 therein. As set out above, the cavity 56 includes internal threading 58 therein corresponding to the external threading 32 on the cylindrical portion 30. A distal or bottom end of the cavity 56 tapers along a conical profile 60 corresponding to the conical end surface 36 of the first body. A passage 70 extends from the conical profile 60 to the second end 54 of the first body along the axis 18.

The second end 54 includes a connector 80 for connection to a standard oxygen tubing 6 extending to the user 8. As illustrated, the connector 80 may include a male connector 82 adapted to be received within the tube 6 with an annular ridge 84 therearound and a threaded cylinder 86 adapted to receive a collar (not shown) on the tube 6. It will also be appreciated that other tube connector types may also be utilized. The exterior surface of the second body 50 may also include ridges, grooves 88 or any other surface treatment to assist gripping by a user.

In operation, a user 8 may connect oxygen tubes 6 to connectors the apparatus 10 such that the passages 40 and 70 of the apparatus are fluidically inline with the oxygen tubing 6. When a user wishes to adjust the flow rate of oxygen therethrough, they may rotate the second body 14 relative to the first body 12. Such rotation will engage the threading 38 and 50 so as to displace the cylindrical portion 30 within the cavity 56. Such longitudinal movement will adjust the gap distance between the conical end profile 34 and the conical profile 60. As such gap is adjusted, a greater or lesser flow rate of oxygen will be permitted therethrough.

As illustrated in FIG. 3, one or both of the cylindrical portion 30 or the cavity 56 may include an annular groove 36 for receiving a seal therein. Optionally, the second body 14 may include a swivel end connection therein. In particular as illustrated in FIGS. 2 and 3, the end connector 80 may be located on a swivel body 90, extending between first and second ends 92 and 94, respectively. The first end 92 may include a cavity 96 therein adapted to receive an end protrusion from the main portion of the second body 14. The cavity and protrusion may include seals, ridges or bearings so as to permit rotation of the swivel body 90 relative to the second body 14. It will be appreciated that the swivel body 90 permits the rotation of the second body 14 relative to the first body without introducing a twist into the oxygen tube 6.

Furthermore, the apparatus may include one or more protrusions extending radially from the cavity 56 or the cylindrical body 30 which are engaged upon one of a plurality of corresponding ridges or grooves in the other of the cavity or the cylindrical body so as to provide a hepatic feedback to a user as the second body 14 is rotated relative to the first body 12. Such feedback will provide an indication of the level to which the apparatus is opened and therefore the relative oxygen flow rate that can be expected at that level.

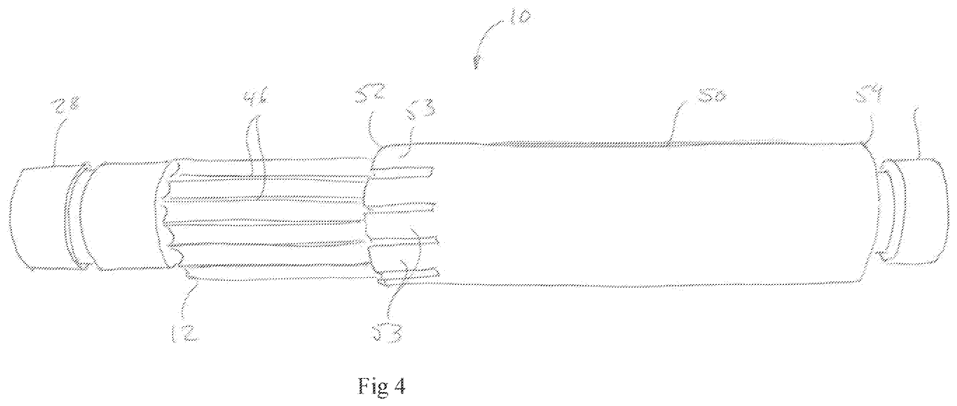

According to a further embodiment, as illustrated in FIG. 4, the first end 52 of the second body 50 may include a plurality of flexible fingers 53 having a protrusion (not shown) at a distal end thereof extending inwards. As the second body 50 is rotated relative to the first body, the protrusions may successively bear against the ridges 46 on the first body thereby providing a feedback to a user of the rotation as well as preventing unwanted rotation therebetweeen.

As illustrated in FIG. 3, the cavity 56 may include a radially inwardly extending wall 100 positioned to be received within an annular groove 102 in the outer surface of the cylindrical portion 30 on the first body 12. As illustrated, the annular groove 102 may have a greater length than the wall 100 so as to provide extended and retracted limits to the movement of the first portion relative to the second portion.

While specific embodiments have been described and illustrated, such embodiments should be considered illustrative only and not as limiting the disclosure as construed in accordance with the accompanying claims.

What is claimed is:

1. An apparatus for controlling the flow of oxygen to a patient comprising:
an outer housing extending between first and second ends having a passage therethrough, the housing comprising:
a first portion extending from the first end towards the second end, and
a second portion extending from the second end towards the first end
wherein the second portion is threadably rotatable relative to the first portion so as to longitudinally displaced relative to the first portion under rotation thereof so as to adjust at least a portion of the passage extending therethrough adjusting a flow capacity of the passage; and
connectors at each of the first and second ends operable to connect to oxygen conduits to fluidically connect the oxygen conduits to the passage through the housing.

2. The apparatus of claim 1 wherein the second portion includes a swivel and a central portion, wherein the central portion is threadably rotatably connected to the first portion and the swivel includes the connector at the second end of the apparatus.

3. The apparatus of claim 2 wherein the swivel and the central portion include the passage extending therethrough.

4. The apparatus of claim 3 wherein the passage is sealed at the connection between the swivel and the central portion.

5. The apparatus of claim 1 wherein the first portion includes a protrusion sized to be threadably received within the second portion.

6. The apparatus of claim 5 wherein the protrusion on the first portion includes threading on the exterior thereof.

7. The apparatus of claim 5 wherein the protrusion includes a conical distal end.

8. The apparatus of claim 5 wherein the second portion includes a cavity therein corresponding to and sized to receive the protrusion of the first portion therein.

9. The apparatus of claim 8 wherein the cavity includes a bottom end having a shape corresponding to the distal end of the protrusion.

10. The apparatus of claim 8 wherein the passage extends through the second portion to the cavity.

11. The apparatus of claim 1 wherein the passage extends along a central axis of the first portion.

12. The apparatus of claim 8 wherein the passage is be obstructed at a distal end of the protrusion proximate to the bottom end of the cavity.

13. The apparatus of claim 12 wherein the passage includes at least one radially extending passage to an exterior of the protrusion within to the bottom end of the cavity.

14. The apparatus of claim 13 wherein at least one radially extending passage is positioned to not overlap with the passage through the second portion.

15. The apparatus of claim 13 wherein longitudinal displacement of the first portion relative to the second portion varies the distance between the protrusion at the at least one radially extending passage and the bottom end of the cavity.

16. The apparatus of claim 1 wherein connectors comprise a protrusion sized to be received within an oxygen tube and a threaded collar adapted to secure the oxygen tube thereto.

17. The apparatus of claim 1 wherein one of the first or second portions includes at least one ridge extending therefrom adapted to be engaged by a corresponding indexing member extending from the other of the first or second portions.

18. A method of adjusting airflow to a patient comprising:
connecting a housing in line with oxygen tubes to the patient;
providing the oxygen tubes with a source of oxygen; and
rotating a second portion of the housing relative to a first portion of the housing to adjust a restriction in a passage extending through the housing.
